(19) United States
(12) Patent Application Publication
(10) Pub. No.: US 2019/0105374 A1
(43) Pub. Date: Apr. 11, 2019

(54) MEDICAL DEVICE COMPRISING COLLAGEN-VI

(71) Applicant: DENTSPLY SIRONA Inc., York, PA (US)

(72) Inventors: Matthias Mörgelin, Mölndal (SE); Christina Gretzner, Göteborg (SE)

(73) Assignee: DENTSPLY SIRONA Inc., York, PA (US)

(21) Appl. No.: 16/211,914

(22) Filed: Dec. 6, 2018

Related U.S. Application Data
(63) Continuation of application No. 14/313,347, filed on Jun. 24, 2014.

(30) Foreign Application Priority Data
Jun. 24, 2013 (EP) 13173411.3

(51) Int. Cl. A61K 38/39; C09D 189/06; A61L 31/10; A61L 27/34; A61L 27/24; A61L 31/04

(52) U.S. Cl. CPC A61K 38/39; C09D 189/06; A61L 31/10; A61L 2430/12; A61L 27/24; A61L 31/044; A61L 2430/02; A61L 27/34

(57) ABSTRACT

A medical device intended for insertion into a living body, which includes a non-biodegradable substrate having a tissue contact surface, wherein said tissue contact surface is at least partially coated with microfibrils of collagen VI.

### CROSS-REFERENCE TO RELATED APPLICATIONS

This patent application is a continuation of U.S. application Ser. No. 14/313,347 filed on Jun. 24, 2014 which claims the benefit of and priority to EPC Application Ser. No. 13173411.3, filed on Jun. 24, 2013, which is herein incorporated by reference for all purposes.

### FIELD OF THE INVENTION

The invention relates to a non-biodegradable medical device intended for insertion into a living body, the medical device comprising a tissue contact surface coated with collagen VI. The invention also relates to methods of manufacturing such devices.

### BACKGROUND

Implantable medical devices may be used for treatment, curing or remedy of many diseases and conditions in a patient's body. Implantable medical devices may be used for replacing a part of the body (e.g. dental and orthopaedic implants, intraocular lenses), or may be used to correct or restore the structure of an internal tissue or organ (e.g. vascular stents). Implantable medical devices may also be used as drug delivery vehicles.

For example, dental implant systems are widely used for replacing damaged or lost natural teeth. In such implant systems, a dental fixture (screw), usually made of titanium or a titanium alloy, is fixed in the jawbone of the patient in order to replace the natural tooth root. An abutment structure is then attached to the fixture in order to build up a core for the part of the prosthetic tooth protruding from the bone tissue, through the soft gingival tissue and into the mouth of the patient. On said abutment, the prosthesis or crown may finally be seated.

For any type of medical device intended for contact with living tissue, biocompatibility is a crucial issue. The risk for foreign body reaction, clot formation and infection, among many other things, must be addressed and minimized in order to avoid adverse effects, local as well as systemic, which may otherwise compromise the health of the patient and/or lead to failure of the device. This is particularly the case for permanent implants. Furthermore, healing or regeneration of tissue around an implant is often vital in order to secure the implant and its long-term functionality. This is especially important for load-bearing implants such as dental or orthopedic implants.

For dental fixtures, a strong attachment between the bone tissue and the implant is necessary. For implants intended for contact with soft tissue, such as abutments which are to be partially located in the soft gingival tissue, also the compatibility with soft tissue is vital for total implant functionality. Typically, after implantation of a dental implant system, an abutment is partially or completely surrounded by gingival tissue. It is desirable that the gingival tissue should heal quickly and firmly around the implant, both for medical and aesthetic reasons. A tight sealing between the oral mucosa and the dental implant serves as a barrier against the oral microbial environment and is crucial for implant success. This is especially important for patients with poor oral hygiene and/or inadequate bone or mucosal quality. Poor healing or poor attachment between the soft tissue and the implant increases the risk for infection and peri-implantitis, which may ultimately lead to bone resorption and failure of the implant.

There are several strategies for increasing the chances of a successful implantation of a medical device, for example enhancing the rate of new tissue formation and/or, in instances where tissue-implant bonding is desired, enhancing the rate of tissue attachment to the implant surface, or by reducing the risk for infection. Enhancement of new tissue formation may be achieved for example by various surface modifications and/or deposition of bioactive agents on the surface. The risk of infection in connection with dental implants is today primarily addressed by preventive measures, such as maintaining good oral hygiene. Once a biofilm is formed on the surface of a dental implant, it is difficult to remove it by applying antibacterial agents. In the case of infection in the bone or the soft tissue surrounding a dental implant (peri-implantitis), mechanical debridement is the basic element, sometimes in combination with antibiotics, antiseptics, and/or ultrasonic or laser treatment.

There is a need in the art for better ways of preventing infections at the site of an implanted medical device.

### SUMMARY OF THE INVENTION

It is an object of the present invention to at least partially overcome this problem, and to provide a medical device, such as an implant, having a surface, which reduces the risk for infection upon contact of the medical device with living tissue.

According to a first aspect of the invention, this and other objects are achieved by a medical device intended for insertion into a living body, the medical device comprising a non-biodegradable substrate having a tissue contact surface, wherein the tissue contact surface is at least partially coated with microfibrils of collagen VI.

Collagen is a protein that forms a major component of the extracellular matrix of many tissues and organs. There are at least 28 different types of collagen found in various tissues. Collage type VI (also denoted “collagen VI”, “collagen-VI” or “type VI collagen”) is a ubiquitous component of the mammalian extracellular matrix. As used herein “collagen VI microfibril” or “microfibrils of collagen VI” refers to a filament structure formed of collagen VI molecule tetramers aggregated end-to-end. Such microfibrils may have a width in the range of from 1 to 50 nm, for example from 5 to 20 nm, and a length in the range of 0.1 to 10 μm, for example from 0.5 to 5 μm.

The inventors have found that a biomaterial coated with collagen VI microfibrils provides antimicrobial properties against aerobic and anaerobic human pathogens. Notably, the antimicrobial effect was exerted by collagen VI present on the surface and likely not involving any significant release of collagen VI from the surface. Hence, the antimicrobial surface may better resist attenuation compared to devices releasing antimicrobial substances. Experimental results suggest that the surface may retain an antimicrobial effect over an extended period of time. The medical device according to embodiments of the invention thus offers better prevention of infections following surgical implantation of an implant or medical device into a patient.

Furthermore, a synergistic effect between the antimicrobial properties of collagen VI and the innate immune system, especially neutrophils, was seen. This was surprising; at most, the recruitment of polymorphonuclear neutrophils (PMNs) to remove the already dead bacteria had been expected, since the main biological function of PMNs is to fagocyte, destroy and discard bacteria. Instead, it was unexpectedly found that PMNs were stimulated to produce neutrophil extracellular traps (NETs) to entrap the bacteria on the surface, such that the bacteria were prevented from further spreading and also rapidly killed by collagen VI. Additionally, bacterial killing by collagen VI is surprisingly enhanced by the presence of PMN and their released proteases. Not wishing to be bound by any particular theory, it is believed that this PMN stimulating effect may, at least partly, be due to the microfibrillar structure of the collagen VI, which includes intact N- and C-terminal domains, is believed to imitate the natural biological environment of a wound, and might also benefit from yet unknown immune and inflammatory processes.

Hence, in situations where neutrophils would come in contact with a collagen VI coated medical device, such as during surgery or any procedure that causes bleeding and/or inflammation at the site of an implant or a medical device, the antibacterial effect will be further enhanced by using a medical device according to the present invention.

Typically, the collagen VI may be present as native microfibrils, with preserved N- and C-terminal globular domains.

In embodiments of the invention, the non-biodegradable substrate comprises a biocompatible material selected from metallic, ceramic or plastic materials. For example, the non-biodegradable substrate comprises a metallic material selected from the group consisting of titanium, zirconium, hafnium, vanadium, niobium, tantalum, cobalt and iridium, and alloys thereof. In other embodiments, the substrate may comprise a ceramic material, for example zirconia.

In embodiments of the invention, the medical device may be a surgical implant, intended for implantation into hard tissue such as bone, and/or soft tissue.

In embodiments of the invention, the medical device may be a load-bearing implant.

For instance, the medical device may be a dental implant or a part thereof, such as a dental fixture, a dental abutment or a one-piece dental implant. In other instances, the medical device may be a bone anchored hearing device. Alternatively, the medical device may be an orthopaedic implant.

In embodiments of the invention, the medical device may be a stent or a shunt.

In embodiments of the invention, the tissue contact surface of the medical device may be coated with a layer of collagen VI. Optionally, the collagen VI may be attached to the surface via linker molecules. A layer of collagen VI may have a layer thickness in the range of from 1 nm to 50 nm, for example from 5 nm to 50 nm. The layer may be discontinuous, i.e., not completely covering the underling surface.

In another aspect, the invention provides a method of coating a surface of a medical device, comprising

(i) providing a non-biodegradable substrate having a tissue contact surface;

(ii) optionally attaching linker molecules to said tissue contact surface;

and

(iii) contacting at least part of said tissue contact surface with a solution comprising microfibrils of collagen VI to attach said microfibrils of collagen VI to said surface and/or said linker molecules.

The linker molecules may comprise poly-L-lysine (PLL).

In embodiments of the invention, step ii) may be performed by

(ii-a) applying a solution comprising the linker molecules and a solvent onto the surface of the article, and

(ii-b) removing said solvent.

Alternatively or additionally, step iii) may be performed by

(iii-a) applying a solution comprising microfibrils of collagen VI and a solvent to said surface;

(iii-b) incubating the substrate having said solution applied to said surface; and

(iii-c) removing said solvent.

Step iii-b) may be performed by keeping the article at a temperature in the range of 4 to 40° C. for at least 10 minutes.

In embodiments of the invention, the solution comprising microfibrils of collagen VI may have a concentration of collagen fibrils in the range of from 10 nM (150 ng/ml) to 10 μM (150 μg/ml), for example from 0.5 to 5 μM, such as from 1 to 2 μM.

Also in the method forming the second aspect of the invention, the collagen VI may preferably be present as native microfibrils.

It is noted that the invention relates to all possible combinations of features recited in the claims.

### BRIEF DESCRIPTION OF THE DRAWINGS

FIG. 1 shows scanning electron micrographs of different surfaces incubated with Streptococcus mitis for 0 hours (left column), 24 hours (middle column) and 48 hours (right column), respectively. The surfaces were the following: a titanium surface (Ti; top row), a Ti surface coated with collagen IV (Ti/cVI; second row from the top), a Ti surface coated with poly-L-lysine, PLL (Ti/PLL; third row from the top) and a Ti surface coated with poly-L-lysine and collagen VI (Ti/PLL/cVI; bottom row). The scale bar represents 10 μm (same scale in all images).

FIG. 2 shows scanning electron micrographs of different surfaces incubated with Actinomyces naeslundii for 0 hours (left column), 24 hours (middle column) and 48 hours (right column), respectively. The surfaces were the following: a titanium surface (Ti; top row), a Ti surface coated with collagen IV (Ti/cVI; second row from the top), a Ti surface coated with poly-L-lysine, PLL (Ti/PLL; third row from the top) and a Ti surface coated with poly-L-lysine and collagen VI (Ti/PLL/cVI; bottom row). The scale bar represents 10 μm (same scale in all images).

FIG. 3 shows scanning electron micrographs of different surfaces incubated with Fusobacterium nucleatum for 0 hours (left column), 24 hours (middle column) and 48 hours (right column), respectively. The surfaces were the following: a titanium surface (Ti; top row), a Ti surface coated with collagen IV (Ti/cVI; second row from the top), a Ti surface coated with poly-L-lysine, PLL (Ti/PLL; third row from the top) and a Ti surface coated with poly-L-lysine and collagen VI (Ti/PLL/cVI; bottom row). The scale bar represents 10 μm (same scale in all images).

FIG. 4 shows scanning electron micrographs of different surfaces incubated with Prevotella intermedia for 0 hours (left column), 24 hours (middle column) and 48 hours (right column), respectively. The surfaces were the following: a titanium surface (Ti; top row), a Ti surface coated with collagen IV (Ti/cVI; second row from the top), a Ti surface coated with poly-L-lysine, PLL (Ti/PLL; third row from the top) and a Ti surface coated with poly-L-lysine and collagen VI (Ti/PLL/cVI; bottom row). The scale bar represents 10 μm (same scale in all images).

FIG. 5 shows scanning electron micrographs of the surfaces Ti/PLL (top row) and Ti/PLL/cVI (bottom row), respectively after 48 hours of incubation with S. mitis (left column), A. naeslundii (second to left column), Fusobacterium nucleatum (second to right column) and Prevotella intermedia (right column), respectively. As can be seen, in the presence of collagen VI (Ti/PLL/cVI) bacterial killing is visible as membrane disruption and cytoplasmic exudation, indicated by white arrowheads. The scale bars represent 2 μm (same scale in all images).

FIG. 6 shows scanning electron micrographs of S. mitis incubated on a collagen VI coated Ti surface (denoted “collagen VI”, top row) and a Ti surface (“control”, bottom row), respectively. Every day a fresh 0.1% solution of bacteria was added to the surfaces. In the presence of collagen VI bacterial growth was significantly inhibited. The scale bar represents 10 μm.

FIG. 7 shows scanning electron micrographs of A. naeslundii incubated on a collagen VI coated Ti surface (denoted “collagen VI”, top row) and a Ti surface (“control”, bottom row), respectively. Every day a fresh 0.1% solution of bacteria was added to the surfaces. In the presence of collagen VI bacterial growth was significantly inhibited. The scale bar represents 10 μm.

FIG. 8 shows S scanning electron micrographs of entrapment of bacteria (S. mitis) in Neutrophil Extracellular Traps (NETs) produced by polymorphonuclear neutrophil (PMNs) on a ceramic dental abutment (top row) and a titanium screw (bottom row). The SEM images show at increasing magnification (scale bars indicating 1 mm, 100 μm, 20 μm and 5 μm) how bacteria are entrapped in NETs ejected by PMN.

FIG. 9 presents a scanning electron micrograph showing entrapment by PMN NETs on a Ti surface coated with collagen VI (Ti/PLL/cVI). The PMN eject NETs to which the bacteria adhere and become entrapped (as indicated by white arrowheads). The scale bar represents 5 μm.

FIGS. 10A and 10B each shows scanning electron micrographs of bacterial entrapment and killing in PMN NETs on the surface of a titanium screw (FIG. 10 A) or a ceramic abutment (FIG. 10B), coated with collagen VI using PLL as linker (cVI; right column)) or without coating (control; left column)) incubated with S. mitis for 0 minutes (top row) and 120 minutes (bottom row), respectively. In the presence of collagen VI the structural integrity of the bacteria is rapidly compromised as evidenced by membrane blebbing (indicated by white arrowheads). Without the collagen VI coating the bacteria remain trapped in NETs but are not killed. The scale bar represents 2 μm.

FIG. 11 schematically depicts the various structures of collagen VI, including polypeptide chains, collagen VI monomers, and a native collagen VI microfibril.

### DETAILED DESCRIPTION

The present inventors have found that a medical device having a tissue contact surface at least partially coated with microfibrils of collagen VI, in particular native microfibrils, may provide a significant antibacterial effect, which is very desirable, in particular for implantable medical devices. Additionally, it was found that collagen VI microfibril coated surfaces exhibited an immune stimulating effect beyond expectation.

As used herein, “collagen VI microfibril” or “microfibrils of collagen VI” refers to a filament structure formed of collagen VI molecule tetramers aggregated end-to-end. The present invention preferably uses native microfibrils, meaning that the microfibil structure corresponds to the native form of collagen VI found in living tissue. In contrast, a non-native microfibril may be partially degraded, e.g. at the N- and/or C-terminal globular domains. Native microfibrils may be isolated from tissue samples using a method as described in Spissinger T, Engel J, Matrix Biol 1995; 14:499-505, using bovine corneal collagenase. Advantageously, this method preserves the globular domains. In contrast, a method using e.g. pepsin cleaves the microfibrils in the globular domains, thus resulting in a partially degraded, non-native collagen VI microfibril.

Directive 2007/47/ec defines a medical device as: “any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings”. In the context of the present invention, only medical devices intended for contact with living tissue are considered, that is, any instrument, apparatus appliance, material or other article of physical character that is intended to be applied on, inserted into, implanted in or otherwise brought into contact with the body, a body part or an organ. Furthermore, said body, body part or organ may be that of a human or animal, typically mammal, subject. Preferably however the medical device is intended for human subjects. Medical devices included within the above definition are for example implants, catheters, shunts, tubes, stents, intrauterine devices, and prostheses.

In particular, the medical device may be a medical device intended for implantation into living tissue or for insertion into the body or a body part of a subject, including insertion into a bodily cavity.

The present medical device may be intended for short-term, prolonged or long-term contact with living tissue. By “short-term” is meant a duration of less than 24 hours, in accordance with definitions found in ISO 10993-1 for the biological evaluation of medical devices. Furthermore, “prolonged”, according to the same standard, refers to a duration of from 24 hours up to 30 days. Accordingly, by the same standard, by “long-term” is meant a duration of more than 30 days. Thus, in some embodiments the medical device of the invention may be a permanent implant, intended to remain for months, years, or even life-long in the body of a subject.

As used herein the term “implant” includes within its scope any device of which at least a part is intended to be implanted into the body of a vertebrate animal, in particular a mammal, such as a human. Implants may be used to replace anatomy and/or restore any function of the body. Generally, an implant is composed of one or several implant parts. For instance, a dental implant usually comprises a dental fixture coupled to secondary implant parts, such as an abutment and/or a restoration tooth. However, any device, such as a dental fixture, intended for implantation may alone be referred to as an implant even if other parts are to be connected thereto.

By “biocompatible” is meant a material which upon contact with living tissue does not as such elicit an adverse biological response (for example inflammation or other immunological reactions) of the tissue.

By “soft tissue” is meant any tissue type, in particular mammalian tissue types, that is not bone or cartilage. Examples of soft tissue for which the medical device is suitable include, but are not limited to, connective tissue, fibrous tissue, epithelial tissue, vascular tissue, muscular tissue, mucosa, gingiva, and skin.

Collagen is a protein that forms a major component of the extracellular matrix of many tissues and organs. There are at least 28 different types of collagen found in various tissues; collagen type I (collagen I) being the most abundant form in bone and connective tissue; collagen type II being predominant in cartilage, collagen III being a major constituent of the blood vessel wall but also present in cartilage, and collagen type IV being a constituent of the basement membrane. An individual collagen molecule consists of three polypeptide chains (also referred to as pro α-chains), each forming an α-helix, closely intertwined in a triple helix configuration. Different types of collagen differ in the amino acid sequences of the polypeptide chains, and also with respect to secondary structure and/or tertiary structure.

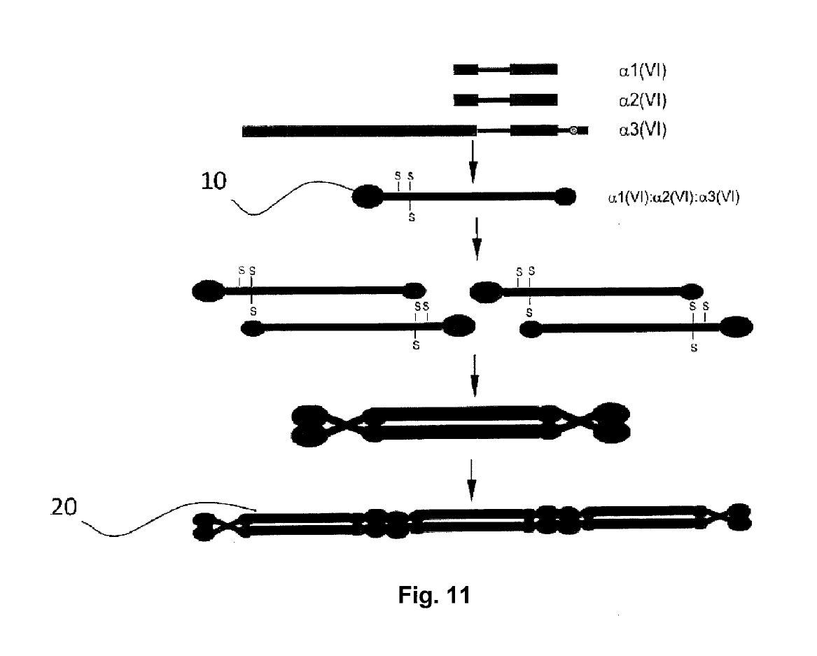

Collage type VI (also denoted “collagen VI”, “collagen-VI” or “type VI collagen”) is a ubiquitous component of the mammalian extracellular matrix. It is present in connective tissues, often associated with basement membranes. As shown in FIG. 11, to form a collagen VI monomer 10, α1, α2, and α3 polypeptide chains assemble in a heterotrimer formation, where additional tissue-specific chains may substitute for the α3 chain in some instances. Four monomers align to a tetramer by lateral association, and a plurality of tetramers aggregate end-on-end to form a microfibril 20 having the shape of a thin, beaded filament. Such microfibrils, also referred to as native microfibrils, typically have a length in the range of from 0.5 to 5 μm and a width of about 10 to 15 nm.

Interestingly, in their biological environment native collagen microfibrils are typically not sensitive to enzymatic degradation. This may be due to the biological role of collagen VI as a biomechanical tissue stabilizer, being important for tissue volume, vascularization and immune cell infiltration.

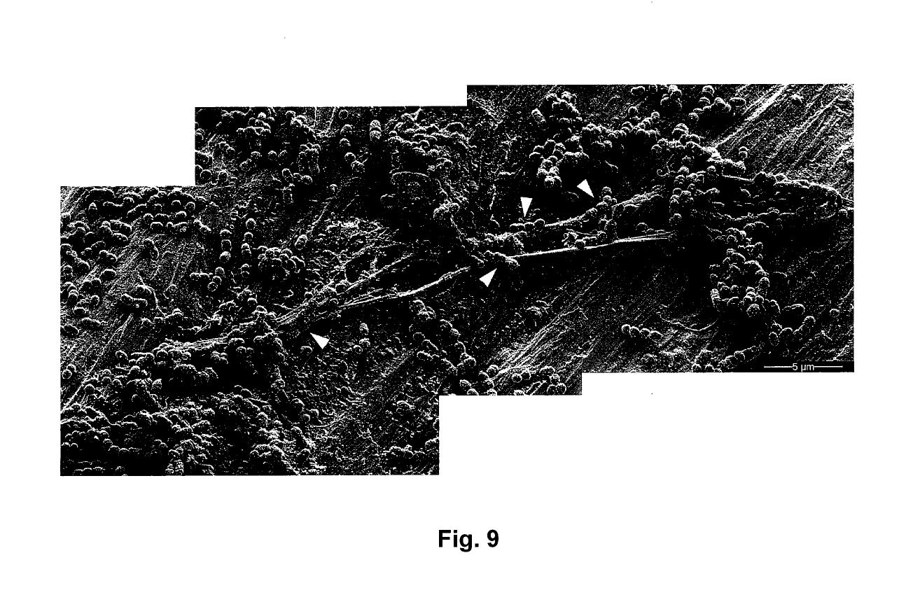

The N- and C-terminal globular domains of collagen VI share homology with von Willebrand factor type A domains (Specks U, Mayer U, Nischt R, Spissinger T, Mann K, Timpl R, Engel J, Chu ML, EMBO J 1992; 11:4281-4290), and collagen VI in solution has been shown to possess an antimicrobial activity against A, C and G streptococci, which are Gram-positive bacteria (Abdillahi S. M., Balvanovic S., Baumgarten M, Mörgelin M., J Innate Immun 2012; 4:371-3762). The present inventors have now shown that collagen VI is not only effective against bacteria when coated onto a device surface, but also has an innate immunomodulating effect, which is likely to be highly beneficial for healing and tissue regeneration. During infection, bacteria stimulate PMN cells to secrete Neutrophil Extracellular Traps (NETs) which entrap and immobilize the bacteria. The recent results show that bacterial killing in NETs is considerably more effective on surfaces which are coated with collagen VI (FIG. 9, 10A-B). Moreover, bacterial killing by collagen VI is surprisingly enhanced by the presence of PMN and their released proteases. Taken together, the results suggest that collagen VI may be beneficial for wound healing in the clinical situation by minimizing the occurrence of bacterial colonization.

In view of these insights and results, the present inventors propose a medical device intended for insertion into a living body, the medical device comprising a non-biodegradable substrate having a tissue contact surface, wherein said tissue contact surface is at least partially coated with collagen VI.

The medical device according to embodiments of the invention may be made of any suitable biocompatible material, e.g. materials used for implantable devices. Typically the medical device comprises a substrate having a tissue contact surface.

By “tissue contact surface” is meant a surface intended for contact (short-term, prolonged, or long-term) with living tissue.

The substrate may for example be made of a biocompatible metal or metal alloy, including one or more materials selected from the group consisting of titanium, zirconium, hafnium, vanadium, niobium, tantalum, cobalt and iridium, and alloys thereof. Alternatively, the substrate of the medical device may be made of a biocompatible ceramic, such as zirconia, titania, shape memory metal ceramics and combinations thereof. In embodiments where the medical device is used as or forms part of a dental abutment, the substrate is preferably made of a metallic material.

In contact with oxygen, the metals titanium, zirconium, hafnium, tantalum, niobium and their alloys instantaneously react to form an inert oxide. Thus, the surfaces of articles of these materials are virtually always covered with a thin oxide layer. The native oxide layer of a titanium substrate mainly consists of titanium(IV) dioxide (TiO₂) with minor amounts of Ti₂O₃, TiO and Ti₃O₄.

Thus, in embodiments where the medical device comprises one or more of titanium, zirconium, hafnium, tantalum, niobium or an alloy of any one thereof, the medical device typically has a native metal oxide surface layer. Such a native metal oxide layer may, in turn, be at least partially covered by a layer of collagen VI microfibrils.

In other embodiments of the present invention, the medical device, in particular the substrate, may be made of a biocompatible polymer, typically selected from the group consisting of polyether ether ketone (PEEK), poly methyl methacrylate (PMMA), poly lactic acid (PLLA) and polyglycolic acid (PGA) and any combinations and copolymers thereof.

In embodiments of the invention, the medical device is intended for short-term, prolonged or long-term contact with living tissue. For example, the medical device of the invention may be an implant, typically intended to temporarily or permanently replace or restore a function or structure of the body.

Typically, at least part of the surface of the medical device is intended for contact with soft tissue, and at least part of this soft tissue contact surface has a coating of collagen VI microfibrils. For example, the medical device may be an implant intended for contact primarily or exclusively with soft tissue, for example a dental abutment. Alternatively, the medical device may be an implant to be inserted partially in bone and partially in soft tissue. Examples of such implants include one-piece dental implants and bone-anchored hearing devices (also referred to as bone anchored hearing aids). Where only part of the implant is intended for contact with soft tissue, it is preferred that the coating comprising collagen-VI is provided at least on a part of a soft tissue contact surface.

The medical device may also be suitable for contact with cartilage.

In other embodiments, the medical device may be intended for contact with bone tissue, e.g. the jawbone, the femur or the skull of a mammal, in particular a human. Examples of such medical devices include dental fixtures and orthopedic implants.

In embodiments of the invention, the tissue contact surface may be a rough surface. The substrate surface roughness, and hence optionally also the surface of the medical device formed by coating with collagen-VI, may have an average surface roughness Rₐ of at least 0.05 μm, typically at least 0.1 μm, for example at least 0.2 μm. Since surfaces having an average surface roughness (Rₐ) of at least 0.2 μm are believed to be more susceptible of biofilm formation, a coating of collagen VI as described herein may be particularly advantageous for medical devices having a surface roughness of at least 0.2 μm, and may be increasingly useful for preventing biofilm formation on medical devices having even higher surface roughness. As an example, a dental abutment comprising a titanium substrate may have a surface roughness of about 0.2-0.3 μm. A coating layer of collagen VI microfibrils may preserve an underlying surface roughness.

Furthermore, in embodiments of the invention, the tissue contact surface of the medical device may comprise at least one additional biomolecule.

The medical device of the invention may be produced by coating the surface with collagen VI microfibrils directly onto the surface or via linker molecule. In embodiments using a linker molecule, the linker molecule is first attached to the surface, and subsequently the collagen microfibrils are attached to said linker molecules. In embodiments using no linker molecule, the surface may however optionally be treated chemically or physically, e.g. in order to clean the surface or to impart a net electrical charge, to enhance attachment of the collagen VI microfibrils. For example, the surface may be subjected to a surface treatment that increases the hydrophilicity of the surface.

After attaching the collagen fibrils, the medical device may optionally be subjected to a mild sterilizing treatment, before use e.g. as an implant or a part thereof.

The collagen VI microfibrils according to embodiments of the present invention may assume any orientation when coated onto a surface, with or without the use of a linker molecule.

For example, the collagen VI microfibrils may be applied to the surface of medical device by applying a solution comprising the collagen microfibrils to the surface. The solution may be applied to the surface by any conventional technique that leaves at least a thin film of solution covering the surface to be coated with collagen VI. Such methods include spraying, pouring and dripping the solution onto the surface, and immersing the surface into the solution.

The solution may be an aqueous solution of collagen VI microfibrils at a concentration in the range of from 10 nM to 10 μM, for example from 0.5 to 5 μM, such as from 1 to 2 μM.

After applying a thin film of a collagen VI solution to the surface, the medical device may be allowed to incubate for a time period of at least 10 minutes, typically at least 30 minutes, for example about 45 minutes, and up to several hours, typically up to 1 hour. Incubation may be carried out at a temperature of 40° C. or less, typically in the range of 4 to 40° C., for example at room temperature (15-25° C.). The medical device may be incubated in a humid chamber. Incubating the device in a humid atmosphere is advantageous because it ensures that the solvent does not evaporate too fast from the surface. A humid chamber as used in embodiments of the invention typically means a closed chamber in which the component is placed, and in which is also present a pool of sterile water or a tissue soaked with sterile water. In an industrial setting the humid chamber may be a controlled chamber with 75-100% humidity. However, it should be noted that a humid chamber is not necessary, and too fast drying of the applied solution may be avoided also at ambient humidity.

After incubation (evaporation of the solvent), the surface is typically washed, e.g. in sterile water or a suitable buffer solution, to remove remaining solution, and may optionally be subjected to a suitable sterilizing treatment, e.g. UV or gamma irradiation or chemical sterilization using ethylene oxide gas.

In embodiments of the invention using a linker molecule, the linker is typically attached to the surface before applying the collagen VI microfibrils. The linker may be attached to the surface by any suitable means, including for example electrostatic interaction, hydrophobic interaction, or covalent binding. In particular, the linker molecule may be attached to the surface via electrostatic interaction. For example, on a surface having a negative electric charge, such as a titanium oxide surface of a titanium article, a positively charged linker molecule such as poly-L-lysine may be attached. If necessary, the surface may be treated or modified by known methods to obtain an electric charge.

The linker molecule may be attached to the surface by applying a solution of the linker molecule onto the surface, preferably so as to completely cover the surface with said solution. Typically, the surface is previously washed e.g. with ethanol, and dried. The solution of linker molecule may be applied by any conventional techniques, such as spraying, pouring or dripping the solution onto the surface or immersing the surface into the solution.

In the case of poly-L-lysine, the solution applied to the surface may be a solution of PLL having a concentration may be in the range of 0.01 to 1 mg/ml, typically about 0.2 mg/ml.

After applying the linker solution to the surface of the medical device, the solvent is removed, leaving the linker molecules attached to the surface. For example, the solvent may be evaporated by treating the medical device at elevated temperature, e.g. in the range of 40 to 60° C., and typically about 60° C. The time required for allowing the solvent to evaporate may be in the range of 10 minutes to 2 hours, typically from 30 minutes to 1 hour.

Optionally, in embodiments of the invention, after applying the linker solution to the surface of the article, the medical device is incubated for a few minutes, e.g. 1-10 minutes, and the linker solution, except those linker molecules that have already bound to the surface, is subsequently washed off by rinsing with a rinsing agent e.g. sterile water, before the article is subjected to elevated temperature as described above. After evaporation or the solvent or the rinsing agent, the surface having attached linker molecules is optionally washed, e.g. with sterile water and dried or allowed to dry.

The collagen microfibrils may be attached to the linker molecules by applying a solution comprising collagen fibrils to the surface coated with the linker molecules according to the description above. The solution comprising collagen fibrils may be applied to the surface by any conventional technique that leaves at least a thin film of solution covering the surface to be coated with collagen fibrils. Such methods include spraying, pouring and dripping the solution onto the surface, and immersing the surface into the solution.

The solution comprising collagen fibrils may be an aqueous solution of collagen VI microfibrils at a concentration in the range of from 10 nM to 10 μM, for example from 0.5 to 5 μM, such as from 1 to 2 μM.

After applying a thin film of collagen solution to the surface, the medical device may be allowed to incubate for a time period of at least 10 minutes, typically at least 30 minutes, for example about 45 minutes, and up to several hours, typically up to 1 hour. Incubation may be carried out at a temperature of 40° C. or less, typically in the range of 4 to 40° C., for example at room temperature (15-25° C.). The medical device may be incubated in a humid chamber. Incubating the device in a humid atmosphere is advantageous because it ensures that the solvent does not evaporate too fast. A humid chamber as used in embodiments of the invention typically means a closed chamber in which the component is placed, and in which is also present a pool of sterile water or a tissue soaked with sterile water. In an industrial setting the humid chamber may be a controlled chamber with 75-100% humidity. However, it should be noted that a humid chamber is not necessary, and too fast drying of the applied solution may be avoided also at ambient humidity.

After incubation (evaporation of the solvent), the surface is typically washed, e.g. in sterile water or a suitable buffer solution, to remove remaining solution, and may optionally be subjected to a suitable sterilizing treatment, e.g. UV or gamma irradiation or chemical sterilization using ethylene oxide gas.

It is envisaged that further modifications could be made to the collagen fibril coating obtained by the method according to the invention. For example, a bioactive substance as described above could be applied to the collagen fibril coating. Additionally of alternatively, the collagen fibrils could be cross-linked after being attached to the surface, e.g. in order to reduce the rate of fibril degradation in vivo after implantation of the medical device.

### Experiments

### A. Material and Methods

### 1. Bacteria

Streptococcus mitis, Actinomyces naeslundii, Fusobacterium nucleatum and Prevotella intermedia were kindly provided by Julia Davies and Gunnel Svensater (Department of Oral Biology, Faculty of Odontology, Malmö University, Malmö, Sweden). S. mitis and A. naeslundii were grown overnight in Todd-Hewitt broth (THB) at 3TC in humid atmosphere containing 5% CO₂. F. nucleatum and P. intermedia were grown in Peptone Yeast Glucose (PYG) medium at 3TC in humid atmosphere under anaerobic conditions.

### 2. Collagen VI

Collagen VI was isolated from bovine cornea by collagenase digestion as described by Abdillahi et al. (2012). Calf eyes were received from the local slaughterhouse. Corneas were cut into pieces and extracted with collagenase, followed by gel filtration with Sepharose CL-2B.

### 3. Coating Titanium

Titanium circles with a diameter of 5 mm were punched out from a foil. First the circles are washed with chloroform and followed by distilled water. After air drying, 50 μL poly-L-lysine (0.2 mg/mL) was applied on desired titanium pieces. Then the pieces have to be incubated at 60° C. for two hours. Afterwards the titanium is washed again in distilled water and air dried. In meantime, 1 mL collagen VI solution is applied into wells of a 12-well plate. Finally desired titanium circles are plunged into the collagen VI solution and incubated over night at 4° C. Following morning, the titanium was air dried and assays were performed.

### 4. Bacterial Adhesion

For adhesion assays S. mitis and A. naeslundii were grown overnight in 10 mL THB, F. nucleatum and P. intermedia were grown in PYG medium under anaerobic conditions and pelleted down at 3500 rpm for 10 minutes at 4° C. on the next day. For the anaerobic species, Fusobacterium nucleatum and Prevotella intermedia, the procedure was performed inside an anaerobic box. Then the pellet was diluted in 10 mL PBST and the OD600 was measured. The OD600 has to be adjusted to 1 and diluted 1:2 in PBST. 500 μL of the bacteria solution were applied into each well with prepared titanium circles inside and incubated at 3TC and 5% CO₂ for 0, 30 and 240 minutes.

The samples were washed with 500 μL PBS three times. PBS was removed and replaced by 500 μL of EM-fix consisting of 2.5% glutaraldehyde in 0.15 M sodium-cacodylate. Samples were incubated in EM-fix overnight. Following steps were performed by an experienced technician. Washing steps with Cacodylate-buffer were performed, followed by a dehydration series. Therefore the samples were incubated for five minutes twice with 50%, 70% and 95% ethanol and with absolute ethanol for 30 minutes and one hour. For drying the samples, ethanol was carried to its critical point to turn into gas by using liquid CO₂. This step was performed three times for ten minutes. Afterwards samples were mounted and coated with gold/palladium 20 nm Agar. Samples were investigated at a scanning electron microscope XL 30 FEG and images were processed by AnalySIS ITEM software.

### 5. Bacterial Killing Assays

5.1 BacLight for FACS

Flow cytometry analysis enables to determine the nature of single cells. In this study BacLight staining was used to stain bacterial cells. That way, it is possible to distinguish between the amount of living and dead bacteria in one population.

Bacteria were grown over night in THB under standard conditions. 1 mL of the overnight culture was transferred to 9 mL fresh THB the next day. Bacteria were incubated under standard conditions, until an OD600 of 0.4 was reached. Bacteria were pelleted down at 3500 rpm and 4° C. for 10 minutes. The supernatant was discarded and the pellet diluted in 10 mL cold TG buffer. The OD was measured and the bacterial solution was pelleted down a second time. After discarding the supernatant the bacterial amount was adjusted to 1% with cold TG buffer. The solution was diluted 1:10 in TG and 20 μL of the solution were transferred to 1.5 mL reaction tubes. 80, 160, 200 or 500 μL collagen VI was applied to the 1.5 mL reaction tubes. For the negative control 80 μL TG buffer were added. For the positive control 300 μL LL-37 were added. Samples were incubated for 0, 2, 24 and 48 hours and stained with 5 μL of a PI and STYO9 diluted 1:100. Samples were measured using BD Accuri C6 flow cytometer.

5.2 Scanning Electron Microscopy (SEM)

With SEM it is possible to investigate the adhesion of bacteria to coated titanium surfaces.

For SEM bacteria were grown overnight to an OD600 of 1. F. nucleatum and P. intermedia were grown in PYG medium under anaerobic conditions, whereas S. mitis and A. naeslundii were grown in THB. The anaerobic species were treated inside an anaerobic box. The cultures were pelleted down and diluted in 10 mL PBST. After the OD600 was adjusted to 1, the bacteria suspension was diluted 1:2 with PBST. 500 μL of this suspension were applied on each well of a 24-well-plate with a coated titanium circle inside. The bacteria were incubated for two hours at 3TC to permit adhesion on the titanium surface. After this time samples were washed with PBS three times and 500 μL THB were added to allow bacterial growth. Bacteria were incubated for 0 minutes, 4, 24 and 48 hours at 3TC. After the incubation, wells were washed three times with 500 μL PBS and bacteria were fixed with EM-fix consisting of 2.5% glutaraldehyde in 0.15 M sodium-cacodylate. Samples were incubated with EM-fix overnight. Following steps were performed by an experienced technician. Washing steps with Cacodylate-buffer were performed, followed by a dehydration series. Therefor the samples were incubated for five minutes twice with 50%, 70% and 95% ethanol and with absolute ethanol for 30 minutes and one hour. For drying the samples, ethanol was carried to its critical point to turn into gas by using liquid CO₂. This step was performed three times for ten minutes. Afterwards samples were mounted and coated with gold/palladium 20 nm Agar. Samples were investigated at a scanning electron microscope XL 30 FEG and images are processed by AnalySIS ITEM software.

### 6. Long-Term Activity of Collagen VI

To find out how long collagen VI is active on titanium surfaces new bacteria were applied to the experimental setting every day new bacteria.

For the experiment titanium was coated as usual. Bacteria were grown over night in THB at standard conditions. The next morning 1 mL of the overnight culture is transferred to 9 mL fresh THB and incubated until the OD600 of the bacteria solution reached 0.4. Bacteria were pelleted down at 3500 rpm for 10 minutes at 4° C. The pellet was resuspended in 10 mL cold TG-buffer and OD as measured. Bacteria were pelleted down again and the bacterial amount was adjusted to 1%. This solution was then diluted 1:10 (0.1% solution). To each well with a titanium slice inside, 500 μL of the bacteria solution was applied and incubated for 0 minutes, 4 h, 24 h, 48 h, 72 h and 96 hours. Then the samples were fixed with EM-fix and prepared for electron microscopy. Every day the bacteria solution was replaced by fresh 0.1% solution.

### 7. Effect of γ-Radiation on Collagen VI

Dental implants are industrially sterilized by γ-radiation. To see, if the radiation has an effect on collagen VI, a long-term study was performed. Titanium was coated as usual and treated with γ-radiation.

### 8. Neutrophil Extracellular Trap (NET) Activation by Oral Bacteria

To investigate how the innate immune system reacts to oral bacteria, growing on dental implants, titanium screw and abutments were coated with either, pLL or pLL/cVI and incubated with S. mitis or A. naeslundii.

8.1 Coating of Dental Implants

Screws and abutments were washed firstly with chloroform and subsequently with deionized water and applied to a 24-well plate. 500 μL of poly-L-Lysine was added until the implants were covered. The implants were incubated at 60° C. until the pLL was dried. Then the implants were washed with deionized water to remove unbound pLL. Screws and abutments that were coated with collagen VI, were applied into an 1.5 mL reaction tube. Collagen VI was added until the implants were covered completely and then incubated at 4° C. overnight. The next day, collagen VI was removed and the implants were air dried.

8.2 Preparation of Bacteria

Bacteria were grown overnight in THB under standard conditions. The next day, 1 mL of bacterial solution was added to 9 mL fresh THB. Bacteria were grown until they reached an OD600 of 0.4. Then they were pelleted down at 3500 rpm and 4° C. for 10 minutes. The supernatant was discarded and the pellet diluted in 10 mL cold TG buffer. The OD was measured and the bacterial solution was pelleted down a second time. After discarding the supernatant the bacterial amount was adjusted to 1% with cold TG buffer. Bacteria were stored on ice until neutrophils were isolated.

8.3 Neutrophil Isolation

For the isolation of neutrophils, 20 mL Polymorph-Prep™ was pipetted into a 50 mL Falcon tube. Blood from healthy donors was collected in Heparin 6 mL tubes and incubated at room temperature for 30 minutes. The polymorph-prep was over layered with 20 mL blood without mixing the fractions. To separate the different blood contents, the falcon tubes were centrifuged for 60 minutes at 500×g and 20° C. After the centrifugation, different layers were visible. The neutrophil layer was removed and transferred into a new falcon tube. Neutrophils are then washed with the double volume of 1×PBS and centrifuged for 10 minutes at 500×g and 20° C. Contaminating erythrocytes are then lysed with 2.7 mL sterile Millipore water for 10 seconds. The reaction was stopped with 300 μL 10×PBS. Volume was adjusted to 15 mL and the samples were centrifuged for 5 minutes at 250×g and 20° C. This step has to be repeated until all erythrocytes are lysed. The pellet was diluted in 500 mL Sodium-medium (containing 5.6 mM glucose, 127 mM NaCl, 10.8 mM KCl, 2.4 mM KH₂PO₄, 1.6 mM MgSO₄, 10 mM Hepes, and 1.8 mM CaCl₂); the pH was adjusted to 7.3 with NaOH) and cells were counted using Luna™ automated cell counter. Amount of cells per sample was calculated and the correspondent volume of neutrophil suspension was added to every 1.5 mL reaction tube containing screws, abutments and bacterial solution. Samples were incubated for 0, 30, 60 and 120 minutes and transferred into 1 mL EM-fix (2.5% glutaraldehyde in 0.15 M sodium-cacodylate). Preparation for SEM was conducted by an experienced technician.

### 9. Statistical Analysis

Data were analyzed by using Excel and GraphPad Prism 6.0. Experiments were performed at least twice independently. Bacterial killing assays using crystal violet were performed in triplets, whereas BacLight viability count for fluorescence microscopy was conducted in duplets. For data of bacterial killing assays using crystal violet and BacLight for fluorescence microscopy a one way analysis of variance (ANOVA) was used. Data received from bacteria incubated in uncoated wells were specified as positive control. The significance is indicated as **** for p<0.0001.

### B. Results

### 1. Bacterial Adhesion

For testing the level of adhesion, bacteria were incubated on coated titanium surfaces and analyzed by scanning electron microscopy. There was some increase of bacterial amount of both Streptococcus mitis and Actinomyces naeslundii detectable after 4 hours of incubation. Between different coatings there is no difference in bacterial adhesion visible. The anaerobic bacterial species Fusobacterium nucleatum and Prevotella intermedia showed a similar degree of adhesion on the surfaces In view of these results, bacteria were incubated for two hours for bacterial killing assays to allow an appropriate level of adhesion.

### 2. Bacterial Killing Assays

2.1 BacLight for FACS

Viability analysis using FACS was used to determine the amount of living bacteria. Propidium iodide (PI) was used to stain dead bacteria. A low STYO9 signal was detected for S. mitis treated with collagen VI. Only after 0 min of incubation approximately 15% of bacteria treated with 160 μL collagen are alive. For the PI signals of bacteria treated with collagen VI increased over time to a maximum of approximately 80% in bacteria treated with 160, 200 and 500 μL.

For A. naeslundii a slight increase of STYO9 stained bacteria can be observed at 48 hours of incubation. For PI the signals of all surface treatment were higher. The highest signals for A. naeslundii treated with collagen VI was be detected after 2 hours of incubation for bacteria treated with 160 μL of cVI. Afterwards the signals decrease again. Hence, bacterial killing caused by collagen VI was stably proved by FACS. The signals for PI staining dead bacteria increased as expected in S. mitis. Within two hours collagen VI killed the pathogens dose independently. The same effect was observed when A. naeslundii was incubated with different amounts of collagen VI. After 2 hours of incubation the maximal bacterial killing was detected. Afterwards the few bacteria that survived start growing again and the signal for PI decreased whereas the signal for STYO9 increased. These results are consistent with the viability results by SEM.

2.2 Scanning Electron Microscopy

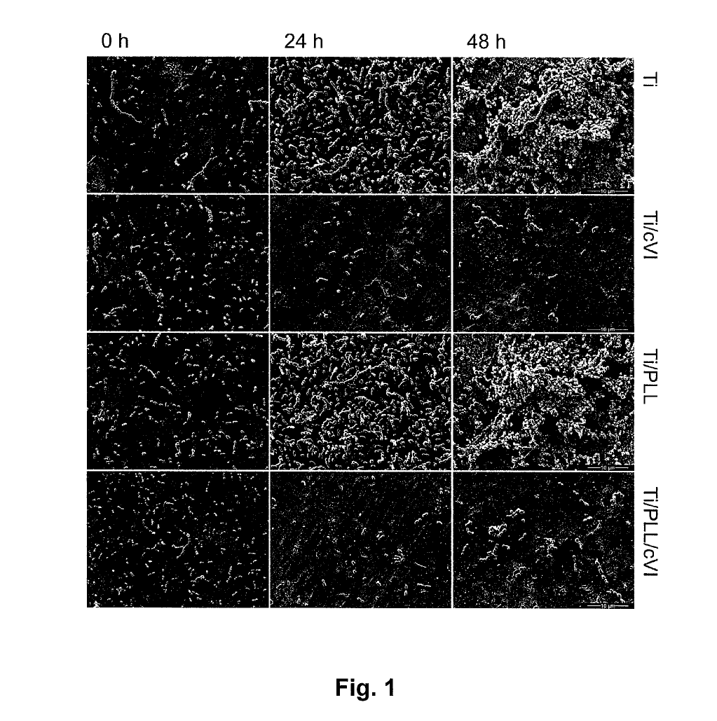

Bacterial killing on titanium surfaces with different coatings was analyzed by scanning electron microscopy during 48 hours. Representative images for the different bacterial species are shown in FIG. 1 to FIG. 4.

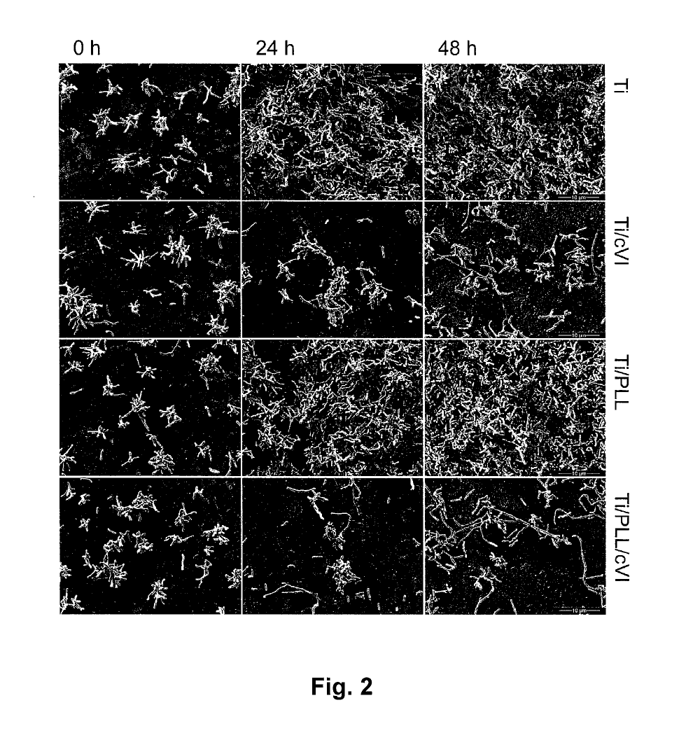

In SEM images an increase of bacterial amount was seen for S. mitis (FIG. 1) and A. naeslundii (FIG. 2) incubated on Ti and Ti/pLL during 48 hours of incubation. Compared to images of S. mitis, A. naeslundii showed a stronger growth. Multilayers were appearing after 24 hours of incubation, and increasing until 48 hours. In contrast, a decrease of bacterial number and an increase in the number of dead, or at least blebbing, bacteria was found when these bacteria were incubated on ti/cVI and ti/pLL/cVI (FIG. 1, FIG. 2). From 24 to 48 hours of incubation, bacterial number increased little. Some colonies were forming during this time, by settling down of bacteria on a layer of dead cells.

Healthy bacteria were observed after four hours of incubation on titanium or titanium coated with pLL. Under these conditions A. naeslundii already starts building multilayer colonies. When this species was treated with collagen VI, the amount of bacteria was decreased after four hours of incubation. Blebbing of vesicular membrane compounds was observed, as well as the ejection of bacterial interior contents.

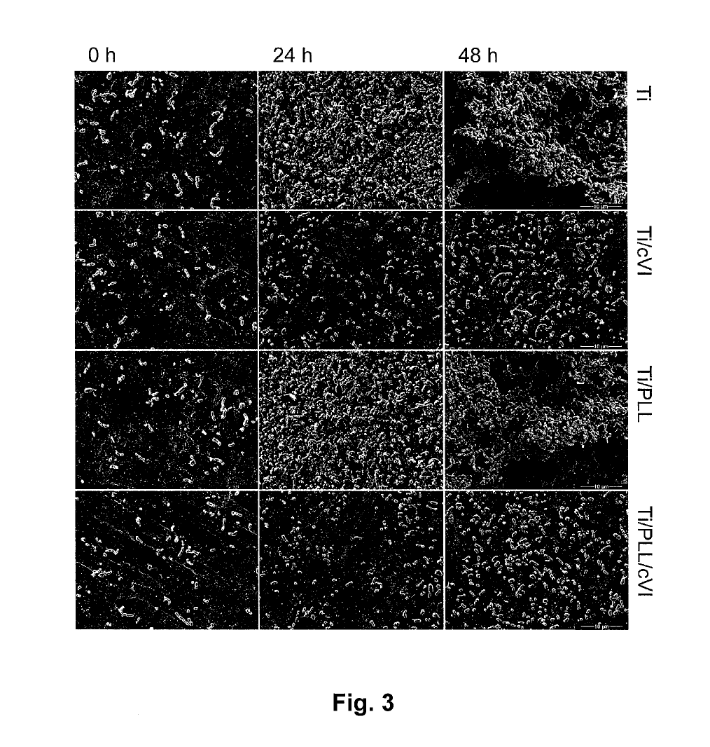

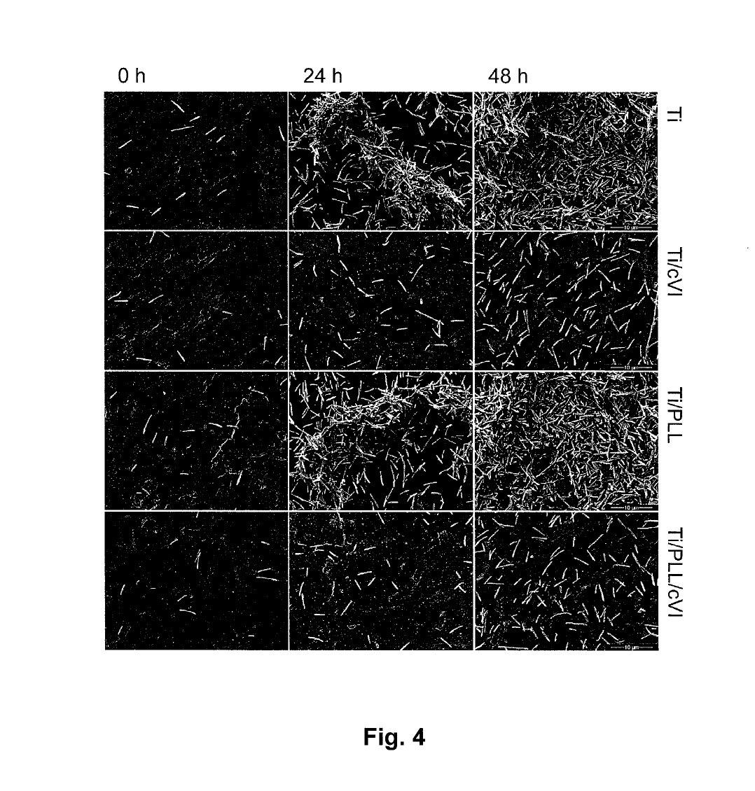

Bacterial killing for Fusobacterium nucleatum and Prevotella intermedia is shown in FIG. 3 and FIG. 4, respectively. For samples not treated with collagen VI, an increase in bacterial amount was seen in both bacteria during 48 hours of incubation. After 24 hours, both species start building massive biofilms similar to those seen for A. naeslundii. In comparison, treatment with collagen VI leads to a strong decrease of bacterial number after already four hours of incubation. Only few bacteria can be detected on the surface. The few bacteria that survived started dividing again, which was seen as increase of bacterial number during 48 hours of incubation. Bacterial growth was decreased compared to the bacteria not treated with collagen VI.

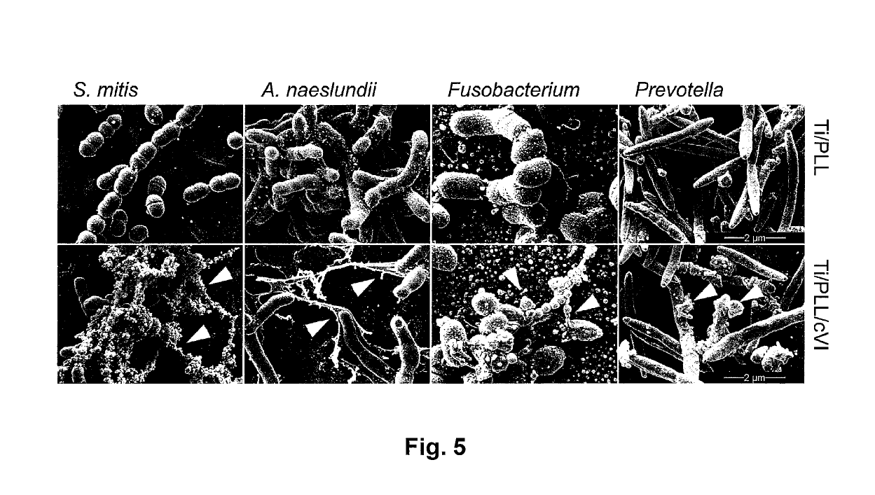

This experiment showed that when S. mitis was treated with collagen VI, surfaces appeared to be empty after 4 hours of incubation compared to S. mitis not treated with collagen VI. At a higher magnification the effects of bacterial killing can be seen for all tested bacterial species (FIG. 5, left column). Bacterial membranes form vesicles and the individual cells seem to be swollen compared to untreated bacteria. Finally, bacteria eject their interior contents, including their DNA. When A. naeslundii in not treated with collagen VI, it forms massive biofilms after 24 hours of incubation. However, when this species is incubated with collagen VI, the surfaces are almost empty after 4 hours of incubation with collagen VI which is related to bacterial killing. After 24 hours few colonies can be detected, which grow until 48 hours of incubation. Compared to untreated A. naeslundii the effect of collagen VI on this pathogens can be seen clearly. In a higher magnification it was clear that A. naeslundii was disrupted by treatment with collagen VI compared to untreated bacteria (FIG. 5, second to left column). The membrane disruption did not occur to equal extent as for S. mitis, but still ejection of bacterial interior contents was observed.

In the anaerobic pathogens the collagen VI coated surfaces appear to be almost empty of bacteria after 24 hours on incubation (FIG. 3, FIG. 4).

In summary, FIGS. 1 to 4 show that during 48 hours of incubation, increasing amounts of growing bacteria are observed in all cases on Ti and Ti/PLL surfaces. After 48 hours, all bacteria have grown to such an extent that they cover the whole surface with a thick layer of biofilm. In contrast, after only four hours of incubation on titanium coated with cVI or pLL/cVI a large amount of dead bacterial as well as blebbing of membrane vesicles and bleeding bacteria was detected, an effect which is even more clearly visible after 24 hours. Bacteria started to eject their interior contents. In contrast, bacteria not incubated with collagen VI looked healthy and had started forming coccids.

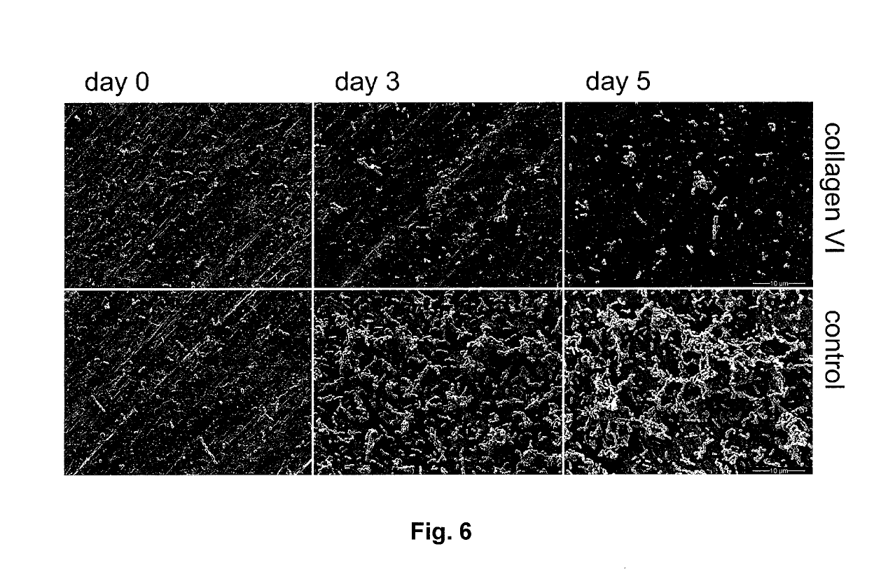

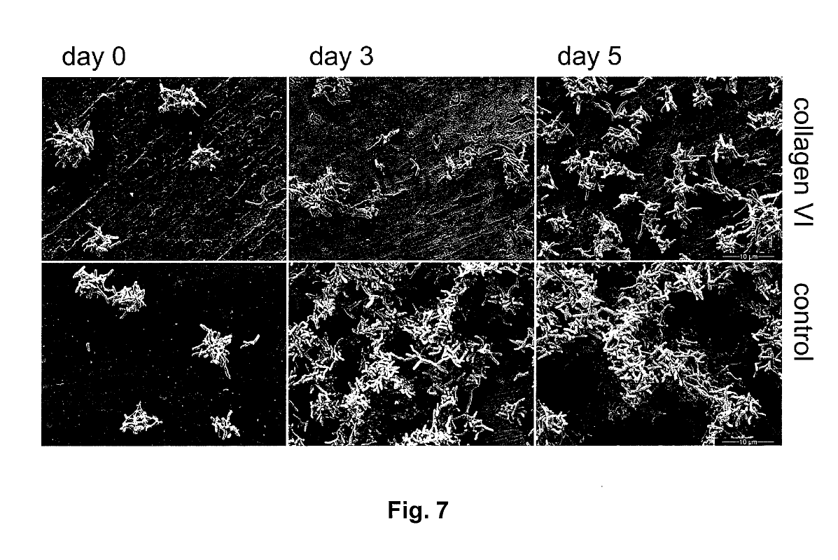

Similar observations were made during a long term study using S. mitis and A. naeslundii. Every day new bacteria were applied to coated titanium surfaces during 5 days. For S. mitis (FIG. 6) as well as for A. naeslundii (FIG. 7) bacterial growth was inhibited when the pathogens were applied on titanium surfaces treated with collagen VI. Untreated bacteria could grow much faster than bacteria growing on collagen VI coated surfaces. Taken together, these experiments demonstrate that the antimicrobial effect of collagen VI is stable at least for 5 days. For dental implants this would mean that coating the implants with collagen VI might prevent infections during the first wave of oral pathogens after surgery.

### 3. Long-Term Activity and γ-Radiation of Collagen VI Coatings

Long-term activity of collagen VI was observed during five days on test surfaces also treated by γ-radiation. Every day, a fresh 0.1% bacterial solution was applied. Without a treatment with collagen VI, bacterial amount increases dramatically in both, S. mitis and A. naeslundii, during 96 hours of incubation. In comparison to that the presence of collagen VI leads to bacterial killing after only two hours of incubation. Some bacteria which survived start dividing afterwards, but not in the same manner as bacteria not treated with collagen VI. If the same experiment is conducted on coated titanium, industrially sterilized by γ-radiation, no difference was observed compared to normal coated titanium. In settings containing collagen VI, bacterial killing was observed after two hours.

### 4. Neutrophil Extracellular Trap (NET) Activation by Oral Bacteria

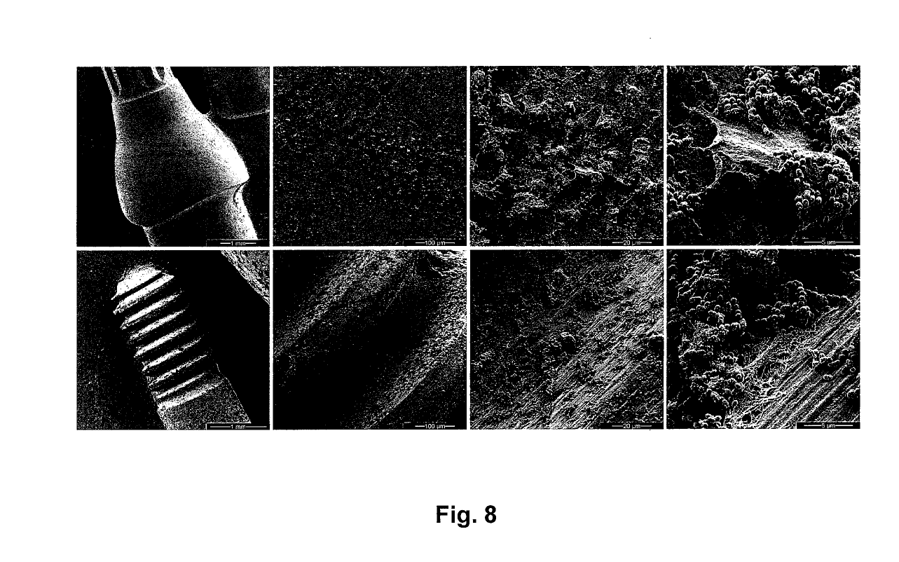

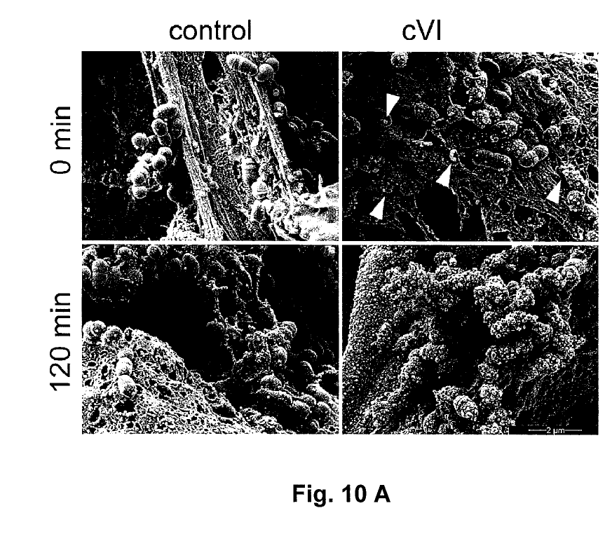

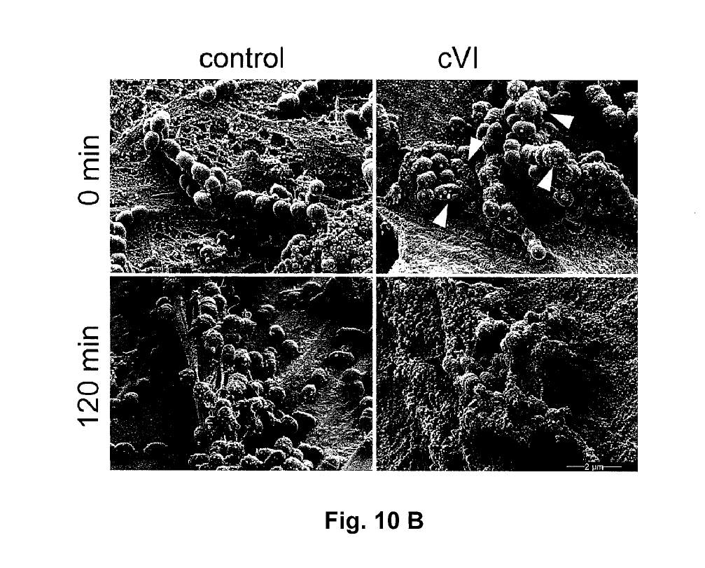

S. mitis and A. naeslundii were incubated on titanium screws (FIG. 8, bottom row) and abutments, respectively (FIG. 8, top row) in independent experiments in the presence of neutrophils. In FIGS. 9 and 10A-B the effect of neutrophils on the oral pathogens can be observed in detail. FIG. 10A (screw) and FIG. 10B (abutment) show killing of S. mitis and NET-formation (NETs indicated by arrows in the figures). Dying bacteria were immediately (0 minutes of incubation) visible in the presence of collagen VI. The effect is enhanced during 120 minutes of incubation, visualized by extensive membrane blebbing and cytoplasmic exudation. When the pathogens are not treated with collagen VI, killing through NETosis can be observed after 120 minutes to a considerably lesser extent (FIG. 10). Similar effects of collagen VI coating can be seen for A. naeslundii. No difference between screw and abutment surfaces was seen.

The innate immune system seems to support and enhance the function of collagen VI or vice versa. During the application of dental implants, the innate immune system gets in contact with the implants and the oral pathogens via the bleeding. For dentistry this means that patients treated with collagen VI coated implants may be considerably better protected against bacterial infections.

### REFERENCES

1. Spissinger T, Engel J, Matrix Biol 1995; 14:499-505
2. Specks U, Mayer U, Nischt R, Spissinger T, Mann K, Timpl R, Engel J, Chu ML, EMBO J 1992; 11:4281-4290
3. Abdillahi S. M., Balvanovic S., Baumgarten M, Mörgelin M., J Innate Immun 2012; 4:371-376

1. A medical device intended for insertion into a living body comprising a non-biodegradable substrate having a tissue contact surface, wherein said tissue contact surface is at least partially coated with microfibrils of collagen VI.

2. A medical device according to claim 1, wherein the collagen VI is present as native microfibrils.

3. A medical device according to claim 2, wherein the native microfibrils comprise preserved N- and C-terminal globular domains.

4. A medical device according to claim 1, wherein said non-biodegradable substrate comprises a biocompatible material selected from metallic, ceramic or plastic materials.

5. A medical device according to claim 4, wherein said non-biodegradable substrate comprises a metallic material selected from the group consisting of titanium, zirconium, hafnium, vanadium, niobium, tantalum, cobalt and iridium, and alloys thereof.

6. A medical device according to claim 4 wherein said non-biodegradable substrate comprises a ceramic material.

7. A medical device according to claim 1, wherein said medical device is a load-bearing implant.

8. A medical device according to claim 1, which is a dental implant or a part thereof.

9. A medical device according to claim 8, wherein said dental implant or part thereof is selected from a dental fixture, a dental abutment and a one-piece dental implant.

10. The medical device according claim 1, which is a bone anchored hearing device.

11. The medical device according to claim 1, which is a stent.

12. The medical device according to claim 1, which is a shunt.

13. The medical device according to claim 1, which is an orthopaedic implant.

14. A medical device according to claim 1, wherein said surface is coated with a layer of collagen VI having a layer thickness in the range of from 1 nm to 50 nm.

15. A medical device according to claim 1, wherein the collagen VI is attached to said surface via linker molecules.

16. A method of coating a surface of a medical device, comprising
(i) providing a non-biodegradable substrate having a tissue contact surface;
(ii) optionally attaching linker molecules to said tissue contact surface; and
(iii) contacting at least part of said tissue contact surface with a solution comprising microfibrils of collagen VI to attach said microfibrils of collagen VI to said surface and/or said linker molecules.

17. A method according to claim 16, wherein step ii) is present and is performed by
(ii-a) applying a solution comprising the linker molecules and a solvent onto the surface of the article, and
(ii-b) removing said solvent.

18. A method according to claim 16, wherein said linker molecules comprise poly-L-lysine (PLL).

19. A method according to claim 16, wherein step iii) is performed by
(iii-a) applying a solution comprising microfibrils of collagen VI and a solvent to said surface;
(iii-b) incubating the substrate having said solution applied to said surface; and
(iii-c) removing said solvent.

20. A method according to claim 19, wherein said solution comprising microfibrils of collagen VI has a concentration of collagen fibrils in the range of from 10 nM to 10 μM.

21. A method according to claim 16, wherein said collagen VI is present as native microfibrils.
